SEQUENCE LISTING (49 sequences)

SEQ ID NO: 1 - DNA, 23 bp, synthetic construct
  misc_feature 1..23: note = primer
  source 1..23: mol_type = other DNA
cccttcactactgttgacttctc

SEQ ID NO: 2 - DNA, 26 bp, synthetic construct
  misc_feature 1..26: note = probe
  source 1..26: mol_type = other DNA
tcacaagaacagatccagtggttgca

SEQ ID NO: 3 - DNA, 24 bp, synthetic construct
  misc_feature 1..24: note = primer
  source 1..24: mol_type = other DNA
ctttgattgcactttggtactctt

SEQ ID NO: 4 - DNA, 20 bp, synthetic construct
  misc_feature 1..20: note = primer
  source 1..20: mol_type = other DNA
gatgagcaggtccgaggtta

SEQ ID NO: 5 - DNA, 23 bp, synthetic construct
  misc_feature 1..23: note = probe
  source 1..23: mol_type = other DNA
ctgcggcgcacccttcagggtct

SEQ ID NO: 6 - DNA, 20 bp, synthetic construct
  misc_feature 1..20: note = primer
  source 1..20: mol_type = other DNA
tcttccaaggcagctttcat

SEQ ID NO: 7 - DNA, 24 bp, synthetic construct
  misc_feature 1..24: note = primer
  source 1..24: mol_type = other DNA
gccagtgtacttcagttggtgcac

SEQ ID NO: 8 - DNA, 29 bp, synthetic construct
  misc_feature 1..29: note = probe
  source 1..29: mol_type = other DNA
tactgtcatttgctcaaagctggctgcca

SEQ ID NO: 9 - DNA, 24 bp, synthetic construct
  misc_feature 1..24: note = primer
  source 1..24: mol_type = other DNA
catttctgccttcatcaccaaaca

SEQ ID NO: 10 - DNA, 22 bp, synthetic construct
  misc_feature 1..22: note = primer
  source 1..22: mol_type = other DNA
aagcatacgtgatggctggtgt

SEQ ID NO: 11 - DNA, 26 bp, synthetic construct
  misc_feature 1..26: note = probe
  source 1..26: mol_type = other DNA
atatgtctcccgccttctgggcatct

SEQ ID NO: 12 - DNA, 25 bp, synthetic construct
  misc_feature 1..25: note = probe
  source 1..25: mol_type = other DNA
catccacggtgcagctggtgacaca

SEQ ID NO: 13 - DNA, 24 bp, synthetic construct
  misc_feature 1..24: note = primer
  source 1..24: mol_type = other DNA
tctaagaggcagccatagggcata

SEQ ID NO: 14 - DNA, 20 bp, synthetic construct
  misc_feature 1..20: note = primer
  source 1..20: mol_type = other DNA
ttctgaagtgcggcctctcc

SEQ ID NO: 15 - DNA, 25 bp, synthetic construct
  misc_feature 1..25: note = probe
  source 1..25: mol_type = other DNA
tcccaaggcaacctcagccatgtcg

SEQ ID NO: 16 - DNA, 24 bp, synthetic construct
  misc_feature 1..24: note = primer
  source 1..24: mol_type = other DNA
cgcttctccttgtggacagaaaac

SEQ ID NO: 17 - DNA, 19 bp, synthetic construct
  misc_feature 1..19: note = primer
  source 1..19: mol_type = other DNA
ttcagtccctgctctcaaa

SEQ ID NO: 18 - DNA, 24 bp, synthetic construct
  misc_feature 1..24: note = probe
  source 1..24: mol_type = other DNA
accgaagaagagaattggacggcc

SEQ ID NO: 19 - DNA, 19 bp, synthetic construct
  misc_feature 1..19: note = primer
  source 1..19: mol_type = other DNA
atcctggtgcctctggtta

SEQ ID NO: 20 - DNA, 22 bp, synthetic construct
  misc_feature 1..22: note = primer
  source 1..22: mol_type = other DNA
ctgtgtcaaccctttcatttac

SEQ ID NO: 21 - DNA, 24 bp, synthetic construct
  misc_feature 1..24: note = probe
  source 1..24: mol_type = other DNA
cattgtccactgcttgtgcccttg

SEQ ID NO: 22 - DNA, 21 bp, synthetic construct
  misc_feature 1..21: note = primer
  source 1..21: mol_type = other DNA
gtgctactcccaaagtcatag

SEQ ID NO: 23 - DNA, 19 bp, synthetic construct
  misc_feature 1..19: note = primer
  source 1..19: mol_type = other DNA
acctccggataggcttcta

SEQ ID NO: 24 - DNA, 22 bp, synthetic construct
  misc_feature 1..22: note = probe
  source 1..22: mol_type = other DNA
atcaaccacacggactgcctgg

SEQ ID NO: 25 - DNA, 19 bp, synthetic construct
  misc_feature 1..19: note = primer
  source 1..19: mol_type = other DNA
ctgtcgtcgaagtggatgc

SEQ ID NO: 26 - DNA, 22 bp, synthetic construct
  misc_feature 1..22: note = primer
  source 1..22: mol_type = other DNA
ctgaagccaggtgactttatac

SEQ ID NO: 27 - DNA, 24 bp, synthetic construct
  misc_feature 1..24: note = probe
  source 1..24: mol_type = other DNA
acctgaatcacatcgagccactga

SEQ ID NO: 28 - DNA, 20 bp, synthetic construct
  misc_feature 1..20: note = primer
  source 1..20: mol_type = other DNA
ttctcgctgaagctgaattt

SEQ ID NO: 29 - DNA, 25 bp, synthetic construct
  misc_feature 1..25: note = primer
  source 1..25: mol_type = other DNA
ctactcctccgtgagcaacatgaac

SEQ ID NO: 30 - DNA, 25 bp, synthetic construct
  misc_feature 1..25: note = probe
  source 1..25: mol_type = other DNA
gcctggggatgaacggcatgaacac

SEQ ID NO: 31 - DNA, 20 bp, synthetic construct
  misc_feature 1..20: note = primer
  source 1..20: mol_type = other DNA
gccgccgacatgctcatgta

SEQ ID NO: 32 - DNA, 25 bp, synthetic construct
  misc_feature 1..25: note = primer
  source 1..25: mol_type = other DNA
taatgagagtgagggaatacctttg

SEQ ID NO: 33 - DNA, 22 bp, synthetic construct
  misc_feature 1..22: note = probe
  source 1..22: mol_type = other DNA
ggcgtgtgtcctttggtgggca

SEQ ID NO: 34 - DNA, 25 bp, synthetic construct
  misc_feature 1..25: note = primer
  source 1..25: mol_type = other DNA
aggcaagttttcatcaaatagttca

SEQ ID NO: 35 - DNA, 28 bp, synthetic construct
  misc_feature 1..28: note = primer
  source 1..28: mol_type = other DNA
accagtgaagaggtcattattcgagaca

SEQ ID NO: 36 - DNA, 30 bp, synthetic construct
  misc_feature 1..30: note = probe
  source 1..30: mol_type = other DNA
cctgttctccctgtcaccctgcagtgtaac

SEQ ID NO: 37 - DNA, 26 bp, synthetic construct
  misc_feature 1..26: note = primer
  source 1..26: mol_type = other DNA
tatgtaagggtgtgagagctggaggt

SEQ ID NO: 38 - DNA, 25 bp, synthetic construct
  misc_feature 1..25: note = primer
  source 1..25: mol_type = other DNA
tgtgacaaggaatatgtgagcctgg

SEQ ID NO: 39 - DNA, 30 bp, synthetic construct
  misc_feature 1..30: note = probe
  source 1..30: mol_type = other DNA
cctgaagatgcatattcggacccacacatt

SEQ ID NO: 40 - DNA, 22 bp, synthetic construct
  misc_feature 1..22: note = primer
  source 1..22: mol_type = other DNA
cgcagatcttgcaaacacaagg

SEQ ID NO: 41 - DNA, 22 bp, synthetic construct
  misc_feature 1..22: note = primer
  source 1..22: mol_type = other DNA
tgacgtccagactccgcttcat

SEQ ID NO: 42 - DNA, 24 bp, synthetic construct
  misc_feature 1..24: note = probe
  source 1..24: mol_type = other DNA
gctgcggccgattgtgaacatgga

SEQ ID NO: 43 - DNA, 22 bp, synthetic construct
  misc_feature 1..22: note = primer
  source 1..22: mol_type = other DNA
acgttctggctcccacgacgta

SEQ ID NO: 44 - DNA, 23 bp, synthetic construct
  misc_feature 1..23: note = primer
  source 1..23: mol_type = other DNA
atgttgacaatgcgtctctggca

SEQ ID NO: 45 - DNA, 29 bp, synthetic construct
  misc_feature 1..29: note = probe
  source 1..29: mol_type = other DNA
tgaccttgaacgcaaagtggaatctttgc

SEQ ID NO: 46 - DNA, 27 bp, synthetic construct
  misc_feature 1..27: note = primer
  source 1..27: mol_type = other DNA
atttcctcttcgtggagtttcttcaaa

SEQ ID NO: 47 - DNA, 22 bp, synthetic construct
  misc_feature 1..22: note = primer
  source 1..22: mol_type = other DNA
ccatcttccaggagcgagatcc

SEQ ID NO: 48 - DNA, 24 bp, synthetic construct
  misc_feature 1..24: note = probe
  source 1..24: mol_type = other DNA
tccacgacgtactcagcgccagca

SEQ ID NO: 49 - DNA, 21 bp, synthetic construct
  misc_feature 1..21: note = primer
  source 1..21: mol_type = other DNA
atggtggtgaagacgccagtg

(19) United States
(12) Patent Application Publication
(10) Pub. No.: US 2026/0226561 A1
(43) Pub. Date: Aug. 6, 2026

(54) A METHOD FOR SORTING COLORECTAL CANCER AND ADVANCED ADENOMA AND USE OF THE SAME

(71) Applicant: INOGENIX Inc., Chuncheon-si (KR)

(72) Inventors: Da som HWANG, Wonju-si (KR); Hyo seok Yang, Chuncheon-si (KR)

(21) Appl. No.: 19/572,000

(22) Filed: Mar. 19, 2026

Related U.S. Application Data
(63) Continuation of application No. 18/088,405, filed on Dec. 23, 2022.

(30) Foreign Application Priority Data
Dec. 31, 2021 (KR) 10-2021-0193852
Dec. 8, 2022 (KR) 10-2022-0170535

(51) Int. Cl. C12Q 1/6886; C12Q 1/6851

(52) U.S. Cl. CPC C12Q 1/6886; C12Q 1/6851; C12Q 2600/112; C12Q 2600/158

(57) ABSTRACT

The present invention relates to a detecting method for colorectal cancer and advanced adenoma group, comprising measuring the relative expression level of MKi67, KRT19, EpCAM, TYMS, PPARG, MCAM, ANKHD1-EIF4EBP3, SNAI2, MMP23B, FOXA2, NPTN, GPR15, TERT, VIM, and ERBB2 genes or proteins encoded by the genes in sample, wherein if the MKi67, KRT19 and EpCAM genes are expressed higher than other genes, it is judged as a normal group, if the TYMS, PPARG, MCAM, and ANKHD1-EIF4EBP3 genes are expressed higher than other genes, it is judged as a colorectal cancer group, if the SNAI2, MMP23B, and FOXA2 genes are expressed higher than other genes, it is judged as an advanced adenoma group or colorectal cancer group, if the NPTN, GPR15, TERT, VIM and ERBB2 genes are expressed higher than other genes, it is judged as an advanced adenoma group.

### CROSS-REFERENCE TO RELATED APPLICATIONS

This application claims the benefit of, and is a continuation of, U.S. Non provisional patent application Ser. No. 18/088,405, filed on Dec. 23, 2022, which claims the priority under 35 U.S.C. § 119 to Korean Patent Application No. 10-2021-0193852, filed on Dec. 31, 2021 and Korean Patent Application No. 10-2022-0170535, filed on Dec. 8, 2022, to the Korean Intellectual Property Office (KIPO). All of the foregoing applications are hereby incorporated by reference as if fully set forth herein in their entirety.

### SEQUENCE LISTING

The content of the electronic sequence listing (OP22-0017HSPCT.xml; Size: 62,449 bytes; and Date of Creation: Mar. 19, 2026) is herein incorporated by reference in its entirety.

### TECHNICAL FIELD

The present invention relates to a method for sorting colorectal cancer and advanced adenoma and use of the same.

### BACKGROUND ART

Colorectal cancer is a cancer that occurs in the colon and rectum, and as of 2020, it is the third most common cancer worldwide and ranks second in cancer-related mortality.

According to the World Colorectal Cancer Incidence Survey conducted by the International Agency for Research on Cancer (IARC) under the World Health Organization (WHO) in 184 countries, the incidence rate of colorectal cancer in Koreans is 45 per 100,000 people. is the highest among the target countries. In addition, according to the data of the National Statistical Office in 2020, it is reported that the third cause of death due to cancer is colorectal cancer. In other words, colorectal cancer is a cancer with a high incidence and mortality rate both in the world and in Korea.

The most important thing in reducing the mortality rate of colorectal cancer is early detection and appropriate treatment of colorectal cancer. According to a recent report, the survival rate of patients reaches 90% when colorectal cancer is detected at stage I, the early stage of colorectal cancer, whereas in the case of stage IV, the late stage of colorectal cancer, it is less than 14%, suggesting that early colorectal cancer diagnosis is very important in improving the survival rate of patients.

Nonetheless, only 37% of colorectal cancers are currently found in stage I, whereas 21% of patients are found in stage IV. Therefore, it can be said that improving the early detection rate of colorectal cancer through regular colorectal cancer screening is very important in reducing colorectal cancer mortality.

Early diagnosis of colorectal cancer is helpful not only in colorectal cancer but also in the detection of polyps or adenomas in the colon. This is related to the mechanism of development of colorectal cancer. In colorectal cancer, it is known that normal colorectal epithelial cells develop into advanced adenoma (AA) for various reasons, and some of them develop into colorectal cancer (CRC). Therefore, regular screening for advanced adenoma and colorectal cancer for early detection/treatment is very important to prevent colorectal cancer. In Korea, a national colorectal cancer screening program is currently being implemented for men and women over the age of 50.

However, the current colorectal cancer health checkup rate in Korea is very low. Currently, in Korea, among the five major cancers (stomach cancer, colorectal cancer, liver cancer, breast cancer, and cervical cancer) health checkup rate (number of examinees compared to the number of test subjects), as of 2019, the screening rate for colorectal cancer is the lowest at 41%. As such, it is believed that the main reason for the lower screening rate for colorectal cancer than other major cancers is the inconvenience of the currently used colorectal cancer screening method.

Regarding the currently used colorectal cancer screening test, the colorectal cancer screening program in Korea conducts fecal occult blood test every year for men and women aged 50 years or older, and if abnormal findings are found in the fecal occult blood test, colonoscopy or colon double contrast examination is recommended.

However, according to a meta-analysis, the sensitivity and specificity of the fecal occult blood test for colorectal cancer were 23-31% and 90-95%, respectively, while the sensitivity for advanced adenoma was only 23-31%, respectively (Niedermaier, T., et al., Eur J Epidemiol, 2017. 32(6): p. 481-493). In addition, since bleeding from colorectal cancer is often intermittent, it is a rule to collect samples for fecal occult blood test three times, once in three consecutive bowel movements, the accuracy of the test may vary depending on whether or not the sample is properly collected. In addition, the subject's compliance with the stool sample is very low.

On the other hand, colonoscopy has very high sensitivity and specificity, and has the great advantage of being able to perform examination and extraction of advanced adenoma during the examination and enabling biopsy using the excised tissue. However, in colonoscopy, the degree of bowel preparation has a very important effect on the accuracy and quality of the examination, so bowel preparation, which is one of the pretreatment procedures, is essential, but the disadvantage is that the process is inconvenient, and the patient's compliance may be reduced.

In addition, there is a problem in that non-advanced adenomas, which have a very low possibility of developing colorectal cancer, may be extracted during colonoscopy, which may cause perforation or bleeding in the colon during the endoscopic procedure. Accordingly, there is a demand in the medical field that it is desirable to perform a colonoscopy only for those who absolutely need a colonoscopy by selecting a risk group having colorectal cancer and advanced adenoma requiring colonoscopy in advance.

A blood test is a representative specimen used for regular examination and is very useful in that it minimizes patient discomfort and enables regular examination. Therefore, the CEA test is used as a screening test for colorectal cancer using blood, but the sensitivity and specificity for detecting colorectal cancer are currently 22-71% and 55-100%, respectively, according to reports, and the sensitivity for detecting advanced adenoma is very high at 14%. It is difficult to use as a screening test for colorectal polyps because it is low.

Therefore, compared to the fecal occult blood test, the test subject's compliance is higher, the pain of the test process is lower than that of the colonoscopy, and the risk of unnecessary perforation or bleeding is low, and the colorectal cancer screening test using blood is highly sensitive to detect colorectal cancer and advanced adenomas. development can be necessary.

### PRIOR PATENT LITERATURE

US Patent Publication No. 20180238893

### DISCLOSURE

### Technical Problem

The present invention solves the above problems and was made by the need, and an object of the present invention is to provide a method for providing information for developing a molecular diagnostic test method for colorectal cancer and advanced adenoma with high sensitivity and specificity based on a blood sample that is relatively easy to extract.

Another object of the present invention is to provide a molecular diagnostic test kit for colorectal cancer and advanced adenoma with high sensitivity and specificity based on a relatively easy-to-extract blood sample.

### Technical Solution

To achieve the above object, the present invention provides a selectively detecting method for colorectal cancer and advanced adenoma group, characterized in that measures the relative expression level of MKi67, KRT19, EpCAM, TYMS, PPARG, MCAM, ANKHD1-EIF4EBP3, SNAI2, MMP23B, FOXA2, NPTN, GPR15, TERT, VIM, ERBB2 genes or proteins encoded by the genes in sample,
wherein if the MKi67, KRT19 and EpCAM genes or proteins encoded by the genes are expressed higher than other genes or proteins encoded by those genes, it is judged as a normal group,
if the TYMS, PPARG, MCAM, and ANKHD1-EIF4EBP3 genes or the proteins encoded by the genes are expressed higher than other genes or proteins encoded by those genes, it is judged as a colorectal cancer group,
if the SNAI2, MMP23B, and FOXA2 genes or the proteins encoded by the genes are expressed higher than other genes or proteins encoded by those genes, it is judged as an advanced adenoma group or colorectal cancer group,
if the NPTN, GPR15, TERT, VIM and ERBB2 genes or the proteins encoded by the genes are expressed higher than other genes or the protein encoded by those genes, it is judged as an advanced adenoma group.

In the method according to the present invention, the method of measuring the expression level of the gene or the protein encoded by the gene can be performed using a known technique, including a known process of isolating mRNA or protein from a biological sample.

The biological sample refers to a sample collected from a living body, and examples of the sample include blood, whole blood, serum, or plasma.

The measurement of the expression level of the gene is specifically to measure the level of mRNA, and methods for measuring the level of mRNA include reverse transcription polymerase chain reaction (RT-PCR), real-time reverse transcription polymerase chain reaction, RNase protection assay, Northern blot and DNA chips, but are not limited thereto.

The protein level may be measured using an antibody. In this case, the protein in the biological sample and an antibody specific thereto form a binding product, that is, an antigen-antibody complex, and the amount of antigen-antibody complex formation can be quantitatively measured through the size of a signal of a detection label. These detection labels may be selected from the group consisting of enzymes, fluorescent substances, ligands, luminescent substances, microparticles, redox molecules, and radioactive isotopes, but are not limited thereto. Assay methods for measuring protein levels include, but are not limited to, Western blot, ELISA, radioimmunoassay, radioimmunoassay, Ouchterlony immunodiffusion assay, rocket immune-electrophoresis, tissue immunostaining, immunoprecipitation assay, complement fixation assay, FACS, and protein chip.

Therefore, the present invention can confirm the mRNA or protein level of a control group and the mRNA or protein level of an individual, such as a test subject, through the detection methods as described above, and colon cancer and/or its precancerous stage can be diagnosed by comparing the expression level with a control group.

In the present invention, the method for measuring the expression of the gene or the protein encoded by the gene is preferably characterized by measuring using primer and probe or using antibody but is not limited thereto.

In one embodiment of the present invention, the primers and probes used are preferably composed of the sequences shown in SEQ ID NOs: 1 to 46 but are not limited thereto.

In addition, the present invention provides a composition for diagnosing colorectal cancer comprising a substance capable of measuring the relative expression levels of TYMS, PPARG, MCAM, and ANKHD1-EIF4EBP3 genes or proteins encoded by the genes.

In one embodiment of the present invention, the substance capable of measuring the relative expression level of the gene is a primer and probe set.

In one embodiment of the present invention, the primer and probe set preferably consists of the sequences set forth in SEQ ID NOs: 1 to 3, SEQ ID NOs: 14 to 16, SEQ ID NOs: 17 to 19, and SEQ ID NOs: 26 to 28, but is not limited thereto.

In addition, the present invention provides a composition for diagnosing an advanced adenoma group comprising a substance capable of measuring the relative expression levels of NPTN, GPR15, TERT, VIM and ERBB2 genes or proteins encoded by the genes.

In one embodiment of the present invention, the substance capable of measuring the relative expression level of the gene is a primer and probe set.

The primer and probe set preferably consists of the sequences shown in SEQ ID NOs: 10 to 13, SEQ ID NOs: 20 to 22, SEQ ID NOs: 35 to 37, SEQ ID NOs: 41 to 43, and SEQ ID NOs: 44 to 46, but is not limited thereto.

In addition, the present invention provides a kit for selectively detecting colorectal cancer and advanced adenomas, comprising:
a substance capable of measuring the relative expression level of proteins encoded by MKi67, KRT19 and EpCAM genes or proteins encoded by the genes,
a substance capable of measuring the relative expression level of TYMS, PPARG, MCAM and ANKHD1-EIF4EBP3 genes or proteins encoded by the genes,
a substance capable of measuring the relative expression level of SNAI2, MMP23B, and FOXA2 genes or proteins encoded by the genes, and
a substance capable of measuring the relative expression levels of NPTN, GPR15, TERT, VIM, and ERBB2 genes or proteins encoded by the genes.

In one embodiment of the present invention, the substance capable of measuring the relative expression level of the gene is a primer and probe set and
in one embodiment of the present invention, the primer and probe set preferably consists of the sequences shown in SEQ ID NOs: 1 to 46 but is not limited thereto.

The present invention will be described below.

In the present invention, primer and probe sequences are provided to indicate the relative expression levels of corresponding biomarkers in blood.

In addition, the present invention provides an artificial intelligence prediction model for colorectal cancer and advanced adenoma screening tests prepared by substituting the expression levels of the 15 markers.

A method for isolating a commonly used full-length RNA (Total RNA) and a method for synthesizing cDNA therefrom can be performed through a known method, and a detailed description of this process can be found in Joseph Sambrook et al., Molecular Cloning, A Laboratory Manual., Cold Spring Harbor Laboratory Press, Cold Spring Harbor, N.Y. (2001); and Noonan, K. F. etc. are disclosed and may be incorporated by reference into the present invention.

The primers of the present invention can be chemically synthesized using the phosphoramidite solid support method, or other well-known methods. Such nucleic acid sequences can also be modified using several means known in the art.

Non-limiting examples of such modifications include methylation, “capping”, substitution of one or more homologs of a natural nucleotide, and modifications between nucleotides, such as uncharged linkages such as methyl phosphonates, phosphotriesters, phosphoramidates, carbamates, etc.) or charged associations (e.g., phosphorothioates, phosphorodithioates, etc.). A nucleic acid can comprise one or more additional covalently linked moieties, such as proteins (e.g., nucleases, toxins, antibodies, signal peptides, L-lysine, etc.), intercalants (e.g., acridine, psoralen, etc.), chelating agents (e.g., metals, radioactive metals, iron, oxidizing metals, etc.), and alkylating agents.

A nucleic acid sequence of the present invention may also be modified with a label capable of providing, directly or indirectly, a detectable signal. Examples of labels include radioactive isotopes, fluorescent molecules, and biotin.

In the method of the present invention, the amplified target sequence may be labeled with a detectable labeling substance. In one embodiment, the label material may be a material that emits fluorescence, phosphorescence, chemiluminescence, or radioactivity, but is not limited thereto. Preferably, the labeling material may be fluorescein, phycoerythrin, rhodamine, lissamine, Cy-5 or Cy-3. When the target sequence is amplified, by labeling the 5′-end and/or 3′-end of the primer with Cy-5 or Cy-3 and performing RT-PCR, the target sequence can be labeled with a detectable fluorescent labeling material.

In addition, when a radioactive isotope such as ³²P or ³⁵S is added to the PCR reaction solution during RT-PCR, the amplification product is synthesized and radioactive is incorporated into the amplification product, so that the amplification product can be radioactively labeled. One or more oligonucleotide primer sets used to amplify the target sequence may be used.

The label provides a signal that can be detected by fluorescence, radioactivity, chromometry, gravimetry, X-ray diffraction or absorption, magnetism, enzymatic activity, mass analysis, binding affinity, hybridization radiofrequency, nanocrystals.

According to one aspect of the present invention, in the present invention, the expression level is measured at the mRNA level through RT-PCR. To this end, novel primer pairs and fluorescently labeled probes that specifically bind to the PPARG and GAPDH genes are required, and in the present invention, corresponding primers and probes specified by specific nucleotide sequences can be used, but are not limited thereto, anything that can specifically bind to these genes to provide a detectable signal to perform RT-PCR can be used without limitation. In the above, FAM and Quen (Quencher) mean fluorescent dyes.

The RT-PCR method applied to the present invention may be performed through a known process commonly used in the art.

The step of measuring the mRNA expression level may be used without limitation as long as it is a method capable of measuring the normal mRNA expression level, and may be performed through radioactivity measurement, fluorescence measurement, or phosphorescence measurement depending on the type of probe label used, but is limited thereto.

As one of the methods for detecting the amplification product, the fluorescence measurement method is to label the 5′-end of the primer with Cy-5 or Cy-3 and perform real-time RT-PCR to label the target sequence with a detectable fluorescent label. And the fluorescence thus labeled can be measured using a fluorescence meter.

In addition, the radioactive measurement method is to add a radioactive isotope such as ³²P or ³⁵S to the PCR reaction solution during RT-PCR to label the amplification product, and then radioactivity can be measured using radioactive measuring instrument, such as a Geiger counter or liquid scintillation counter.

According to a preferred embodiment of the present invention, a fluorescence-labeled probe is attached to the PCR product amplified through the RT-PCR to emit fluorescence of a specific wavelength, and at the same time as amplification, the fluorescence meter of the PCR device measures the genes of the present invention. The mRNA expression level is measured in real time, and the measured value is calculated and visualized through a PC, so that the inspector can easily check the expression level.

According to another aspect of the present invention, the screening kit may be a kit for diagnosing colorectal cancer and colorectal polyps, characterized in that it includes essential elements necessary for carrying out a reverse transcription polymerase reaction. The reverse transcription polymerase reaction kit may include each primer pair specific for the gene of the present invention. The primer is a nucleotide having a sequence specific to the nucleic acid sequence of each marker gene, and may have a length of about 7 bp to 50 bp, more preferably about 10 bp to 30 bp.

Other reverse transcription polymerase reaction kits include a test tube or other suitable container, reaction buffer (with varying pH and magnesium concentration), deoxynucleotides (dNTPs), enzymes such as Taq-polymerase and reverse transcriptase, DNAse, RNAse inhibitors, DEPC-water, sterile water, and the like.

In addition, the kit of the present invention may further include a user guide describing optimal reaction performance conditions.

The guide is a printed matter that explains how to use the kit, e.g., how to prepare a buffer solution, suggested reaction conditions, and the like.

The guide may include a brochure in the form of a pamphlet or leaflet, a label affixed to the kit, and instructions on the surface of the package containing the kit. In addition, the guide may include information disclosed or provided through an electronic medium such as the Internet.

In the present invention, the term “colorectal cancer screening method” is a preliminary step for diagnosis and provides objective basic information necessary for diagnosis of cancer, and clinical judgment or opinion of a doctor is excluded.

The term “primer” refers to a short nucleic acid sequence having a short free 3-terminal hydroxyl group capable of forming base pairs with a complementary template and serving as a starting point for copying the template strand. Primers can initiate DNA synthesis in the presence of reagents for polymerization (i.e., DNA polymerase or reverse transcriptase) and four different nucleoside triphosphates in an appropriate buffer and temperature. The primers of the present invention are sense and antisense nucleic acids having sequences of 7 to 50 nucleotides specific to each marker gene. A primer may incorporate additional features that do not alter the basic properties of the primer that serve as the starting point of DNA synthesis.

The term “probe” is a single-stranded nucleic acid molecule and comprises a sequence complementary to a target nucleic acid sequence.

The term “real-time RT-PCR” is a molecular biological polymerization method that RNA is reverse transcribed into complementary DNA using reverse transcriptase, and then using the prepared cDNA as a template, the target is amplified using target primers and a target probe containing a label, and at the same time, a signal generated from the label of the target probe is quantitatively detected in the amplified target.

A data mining method capable of diagnosing colorectal cancer and advanced adenoma groups through information learning can be used for the prediction of colorectal cancer and advanced adenoma groups of the present invention, and, it can be effectively improved through AI analysis. Therefore, a method capable of measuring the relative expression levels of diagnostic markers for colorectal cancer and advanced adenoma groups and/or an AI analysis method may be preferably used in the method for diagnosing or predicting colorectal cancer and advanced adenoma groups of the present invention.

In the present invention, when AI analysis is used for colorectal cancer and advanced adenomatous group prediction models, various interpretable models can be used without limitation, and linear regression, logistic regression, neural network analysis, decision tree, decision rule, rule fit, support vector Machine-like models are applicable without limitation, and preferred embodiments of the present invention utilize logistic regression analysis, decision trees, neural network analysis, and support vector machines, among others.

Meanwhile, the prediction model of the present invention may include a colorectal cancer and advanced adenoma group diagnosis unit, a classification unit, and a weighting unit. Using the received relative expression level information as input information, the colon-related disease classification unit may perform a process of classifying colon cancer and colon polyps using a neural network as a classifier, and the weighting unit may select colorectal cancer and advanced adenoma groups by assigning weights to classification results.

Neural network analysis according to embodiments of the present invention refers to a system that constructs one or more layers to decide based on a plurality of data. For example, in neural network analysis, the input layer is a layer that inputs relative expression level information of gene markers as data into a neural network analysis model, and the output layer is a layer that gives results that determines the presence or absence of colorectal cancer and advanced adenoma disease patients based on various input information. The hidden layer is a layer that proceeds with the process of determining whether there is a patient by assigning weights to various criteria (gene mutation information).

The method for predicting colorectal cancer and advanced adenoma using an AI analysis technique according to an embodiment of the present invention estimates a neural network analysis model having the number of hidden nodes using an MLP neural network. In addition, among several neural network models built through various variable transformations of input and output variables, the neural network model with the highest accuracy estimated from each model is determined as the final neural network model for colon related disease prediction. The AI analysis may be composed of an input layer, a hidden layer, and an output layer, and the neural network analysis model through the neural network analysis step may be a neural network model having several hidden nodes in several hidden layers.

### Advantageous Effects

As can be seen from the present invention, the present invention can help in screening for colorectal cancer and advanced adenoma by substituting the expression patterns of genetic markers expressed in blood into an artificial intelligence algorithm using a relatively easy-to-extract blood sample.

### DESCRIPTION OF DRAWINGS

FIG. 1 is a diagram showing a heatmap showing the expression pattern of each group of genes,

FIG. 2 is a diagram showing an overview of model construction and performance confirmation of an embodiment of the present invention;

FIG. 3 is a diagram showing the ROC Curve and PR Curve in the test set and

FIG. 4 is a diagram showing an outline of model construction and performance confirmation of a comparative example of the present invention.

### MODE FOR INVENTION

Hereinafter, the present invention will be described in more detail by the following examples. However, the following examples are described with the intention of illustrating the present invention, and the scope of the present invention is not to be construed as being limited by the following examples.

### Example 1: Collection of Clinical Specimens

From 2017 to 2022, Blood samples from subjects scheduled for colonoscopy were collected at the Shinchon Severance Hospital (Approval No. 4-2017-0148), the Gangnam Severance Hospital (Approval No. 3-2017-0024), the Kangbuk Samsung Hospital (Approval No. 2017-02-022-009) in the Department of Gastroenterology, the Health Examination Center of Wonju Severance Christian Hospital (approval number CR319115) with the approval of the Bioethics Review Board (RB) of each institution. A total of 3 ml of blood was collected using a Tempus blood tube (Applied Biosystems®). Subjects were classified as follows through the results of colonoscopy (Table 1)

TABLE 1

| | | No. of |
|---|---|---|
| | | samples |
| Classification | Classification criteria | (persons) |
| Colorectal | As a result of colonoscopy, | 148 |
| cancer | subjects with cancer in the colon | |
| group | | |
| Advanced | As a result of colonoscopy, | 289 |
| adenoma | subjects with advanced adenoma | |
| group | in the colon | |
| Normal | Subjects with no lesions in the | 142 |
| group | large intestine as a result of | |
| | colonoscopy | |

| | |
|---|---|
| Total | 579 |

Table 1 shows the classification of subjects and the number of samples according to colonoscopy results.

### Example 2: Isolation of Total RNA from Blood Specimens

Total RNA is isolated from a blood sample collected with a Tempus tube using the Tempus blood RNA isolation kit (Applied Biosystems®).

### Example 3: CDNA Construction from Isolated Total RNA and gPCR

i. Complementary DNA (cDNA) Synthesis

Isolated total RNA 1.5~4.5 ug, Random primer (3 ug/uL) (Invitrogen) 2.5 uL, dNTP mixture (2.5 mM each) (Intron) 2.5 uL, M-MLV reverse transcription polymerase (200 U/uL) (Invitrogen) 2.5 uL, 10 μL of 5× First-strand buffer (250 mM Tris-HCl) (Invitrogen), and 5 μL of Dithiothreitol (0.1 M) (Invitrogen) were added, and ultrapure water was added to a final volume of 50 μL, and mixed well. The synthetic reaction solution was reacted in a thermocycler (Applied Biosystems) at 25° C., 30 minutes—37° C., 50 minutes—70° C., 15 minutes to synthesize cDNA.

ii. Perform Quantitative Polymerase Chain Reaction (qPCR)

For the composition of the qPCR reaction, added 10 μL of THUNDERBIRD® Probe qPCR Mix (TOYOBO), Forward/Reverse Primer, Probe (10 pmole/uL) 1 μL, and added 2 μL of synthesized cDNA, and add ultrapure water to make the final volume 20 μL, and mixed. The qPCR reaction was performed using CFX96 (Biorad), and the reaction temperature conditions were as follows. After 95° C., 3 minutes, 95° C., 3 seconds—60° C., 30 seconds were repeated 40 times. Each time the annealing process (60° C., 30 seconds) was performed, a process of measuring fluorescence was added to measure the fluorescence value that increased by number of times. A constant fluorescence value was set as the threshold, and the Cq value, which is the number of cycles at the time of reaching the threshold, was derived.

### Example 4: Confirmation of Results and Analysis of Relative Expression of Target Genes

Using the Cq value of the GAPDH gene used as an endogenous control, the relative expression level (2^{-ΔCq}) of the target gene is calculated using the Cq value of the target gene. A list of targeted genes follows (Table 2).

$${2}^{- Δ Cq} = {2}^{- ( target gene Cq - GAPDH gene Cq )} \qquad [ Calculation formula ]$$

TABLE 2

| No. | Blood genetic markers | |
|---|---|---|
| 1 | ANKHD1- | ANKHD1-EIF4EBP3 Readthrough |
| | EIF4EBP3 | |
| 2 | EpCAM | Epithelial Cell Adhesion Molecule |
| 3 | ERBB2 | Erb-B2 Receptor Tyrosine Kinase 2 |
| 4 | FOXA2 | Forkhead Box A2 |
| 5 | GPR15 | G Protein-Coupled Receptor 15 |
| 6 | KRT19 | Keratin 19 |
| 7 | MCAM | Melanoma Cell Adhesion Molecule |
| 8 | MKi67 | Marker Of Proliferation Ki-67 |
| 9 | MMP23B | Matrix Metallopeptidase 23B |
| 10 | NPTN | Neuroplastin |
| 11 | PPARG | Peroxisome Proliferator Activated Receptor |
| | | Gamma |
| 12 | SNAI2 | Snail Family Transcriptional Repressor 2 |
| 13 | TERT | Telomerase Reverse Transcriptase |
| 14 | TYMS | Thymidylate Synthetase |
| 15 | VIM | Vimentin |

Table 2 is a list of target blood genetic markers.

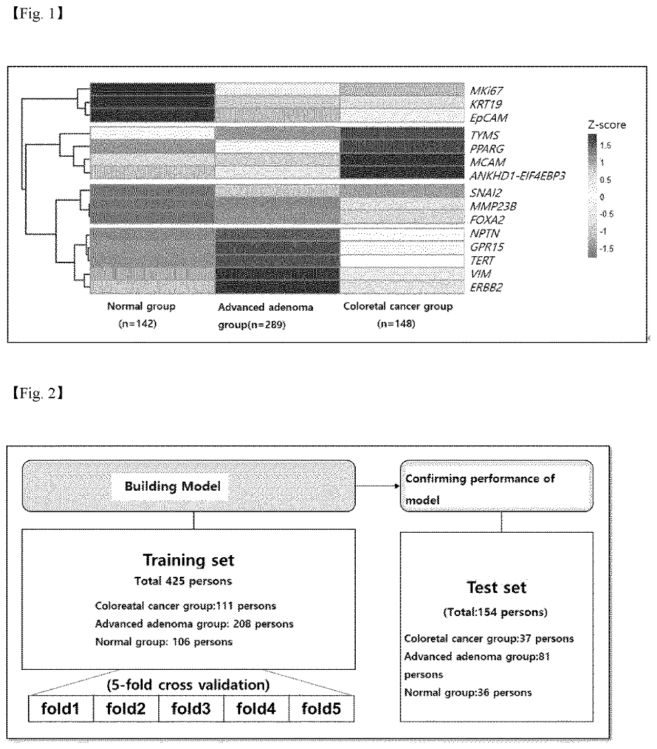

In order to compare the relative expression amount of each gene group, a heatmap based on the average relative expression amount of each gene group was constructed using the pheatmap package (version 1.0.12) of Statistical R software (version 3.6.3) (FIG. 1). When building a heatmap, colors are displayed according to the Z-score, and the Z-score calculation formula for each gene group is as follows. The lower the Z-score, the lower the expression compared to other groups, and the higher the Z-score, the higher the expression compared to other groups.

$$Z - score = ( expression level of the group - average expression level in all groups ) / ( standard deviation between all groups ) \qquad [ Calculation formula ]$$

As a result, 3 genes (MKi67, KRT19, EpCAM) were highly expressed in the normal group compared to other groups and 4 genes (TYMS, PPARG, MCAM, ANKHD1-EIF4EBP3) were highly expressed in the colorectal cancer group compared to other groups, and 3 genes (SNAI2, MMP23B, FOXA2) were highly expressed in the advanced adenoma group and colorectal cancer group compared to other groups, and five genes (NPTN, GPR15, TERT, VIM, ERBB2) were highly expressed in the advanced adenoma group.

### Example 5: Establishment of a Classification Model for the Purpose of Screening for Colorectal Cancer and Advanced Adenoma by Substituting the Relative Expression Level of Target Genes

An artificial intelligence algorithm-based classification model was constructed using the H2O package (version 3.32.1.3) of Statistical R software (version 3.6.3). The production of colorectal cancer and advanced adenoma diagnosis prediction models was based on Deep neural network (DNN), Generalized linear model (GLM), Random Forest (RF), and Gradient boosting machine (GBM) algorithms, and several types of models (GLM, RF, DNN, GBM, stacked ensemble (SE)) was performed by grafting Automated machine learning (AutoML) method to build a model suitable for data, but is not limited thereto.

By dividing the entire sample into a training set and a test set, and by substituting the results of the training set, an artificial intelligence algorithm-based classification model that can distinguish between the colorectal cancer group and the advanced glandular group compared to the normal group is constructed, and the performance of the built model is evaluated using the test set (FIG. 2).

When building a model using a training set, a 5-fold cross-validation technique is applied so that the training set is divided into 5 areas and so at the same time as learning the model, the performance of the model was verified using each area to build a high-performance model.

The performance of the artificial intelligence classification model was judged through the AUROC and AUPRC values of the training set and test set based on the AUROC and AUPRC values, which are representative performance indicators of the classification model. Among them, the model with the best performance was selected based on the performance of the new test set that was not used for model learning.

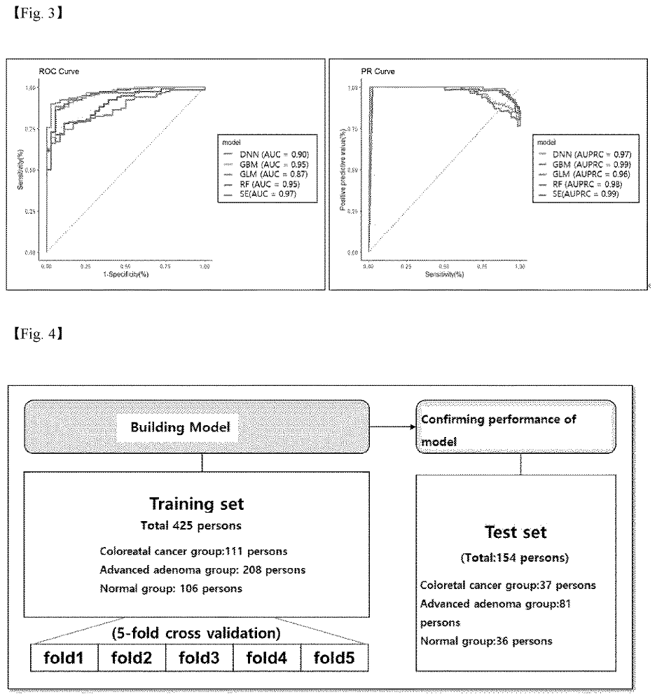

The AUROC and AUPRC values of the GLM, DNN, GBM, and RF models built based on each algorithm and the SE model built through AutoML are as follows (Table 3). As a result, the AUROC and AUPRC indicators were the highest in the SE model based on the test set (FIG. 3).

TABLE 3

| | Training set | | Test set | |
|---|---|---|---|---|
| Model | AUROC | AUPRC | AUROC | AUPRC |
| GLM | 0.91 | 0.97 | 0.87 | 0.96 |
| RF | 0.92 | 0.97 | 0.95 | 0.98 |
| DNN | 0.90 | 0.96 | 0.90 | 0.97 |
| GBM | 1.00 | 1.00 | 0.95 | 0.99 |
| AutoML | 1.00 | 1.00 | 0.97 | 0.99 |
| (SE) | | | | |

Table 3 shows AUROC and AUPRC performance indicators in the training set and test set.

As a result of confirming the sensitivity and specificity of each group in the SE model, as shown in Table 4, the sensitivity to classify the colorectal cancer group was 91.9%, the sensitivity to classify the advanced adenoma group was 92.6%, and the specificity to classify the normal group was 91.7%.

TABLE 4

| | Result of Test set | | | |
|---|---|---|---|---|
| | (Total 154 persons) | | | |
| | Positive | Negative | Sensitivity | Specificity |
| Classification | (persons) | (persons) | (%) | (%) |
| Colorectal | 110 | 8 | 92.4 | |
| cancer group + | | | | |
| Advanced | | | | |
| adenoma | | | | |
| group(n = 118) | | | | |
| colorectal | 34 | 3 | 91.9 | |
| cancer group | | | | |
| (n = 37) | | | | |
| Advanced | 75 | 6 | 92.6 | |
| adenoma | | | | |
| group(n = 81) | | | | |
| Normal group | 3 | 33 | | 91.7 |
| (n = 36) | | | | |

Table 4 shows the sensitivity and specificity results for each group of the SE model.

TABLE 5

| | Primer and | | Primer's and Taqman | PCR |
|---|---|---|---|---|
| Target | TaqMan | Sequence | probe's sequence | product |
| gene | probe | No | (5′ --> 3′) | (bp) |
| PPARG | Forward | 1 | CCC TTC ACT ACT GTT GAC TTC TC | 133 |
| | Taqman | 2 | FAM-TCA CAA GAA CAG | |
| | probe | | ATC CAG TGG TTG CA-BHQ1 | |
| | Reverse | 3 | CTT TGA TTG CAC TTT GGT ACT CTT | |
| KRT19 | Forward | 4 | GAT GAG CAG GTC CGA GGT TA | 96 |
| | Taqman | 5 | FAM-CTG CGG CGC ACC | |
| | probe | | CTT CAG GGT CT-BHQ1 | |
| | Reverse | 6 | TCT TCC AAG GCA GCT TTC AT | |
| EpCAM | Forward | 7 | GCC AGT GTA CTT CAG TTG GTG CAC | 82 |
| | Taqman | 8 | FAM-TAC TGT CAT TTG CTC | |
| | probe | | AAA GCT GGC TGC CA-BHQ1 | |
| | Reverse | 9 | CAT TTC TGC CTT CAT CAC CAA ACA | |
| ERBB2 | Forward | 10 | AAG CAT ACG TGA TGG CTG GTG T | 115 |
| | Taqman | 11 | FAM-ATA TGT CTC CCG CCT | |
| | probe1 | | TCT GGG CAT CT-BHQ1 | |
| | Taqman | 12 | FAM-CAT CCA CGG TGC | |
| | probe2 | | AGC TGG TGA CAC A-BHQ1 | |
| | Reverse | 13 | TCT AAG AGG CAG CCA TAG GGC ATA | |
| MCAM | Forward | 14 | TTC TGA AGT GCG GCC TCT CC | 74 |
| | Taqman | 15 | FAM-TCC CAA GGC AAC | |
| | probe | | CTC AGC CAT GTC G-BHQ1 | |
| | Reverse | 16 | CGC TTC TCC TTG TGG ACA GAA AAC | |
| ANKHD1- | Forward | 17 | TTCAGTCCCTGCTCTCAAA | 108 |
| EIF4EBP3 | Taqman | 18 | FAM-ACCGAAGAAGAGAATTGG | |
| | probe | | ACGGCC-BHQ1 | |
| | Reverse | 19 | ATCCTGGTGCCTCTGGTTA | |
| GPR15 | Forward | 20 | CTG TGT CAA CCC TTT CAT TTAC | 106 |
| | Taqman | 21 | FAM-CAT TGT CCA CTG CTT | |
| | probe | | GTG CCC TTG-BHQ1 | |
| | Reverse | 22 | GTG CTA CTC CCA AAG TCA TAG | |
| MMP23B | Forward | 23 | ACC TCC GGA TAG GCT TCT A | 136 |
| | Taqman | 24 | FAM-ATCAACCACACGGACTGCC | |
| | probe | | TGG-BHQ1 | |
| | Reverse | 25 | CTG TCG TCG AAG TGG ATG C | |
| TYMS | Forward | 26 | CTGAAGCCAGGTGACTTTATAC | 90 |
| | Taqman | 27 | FAM-ACCTGAATCACATCGAGCC | |
| | probe | | ACTGA-BHQ1 | |
| | Reverse | 28 | TTCTCGCTGAAGCTGAATTT | |
| FOXA2 | Forward | 29 | CTA CTC CTC CGT GAG CAA CAT GAA C | 74 |
| | Taqman | 30 | FAM-GCC TGG GGA TGA | |
| | probe | | ACG GCA TGA ACA C-BHQ1 | |
| | Reverse | 31 | GCC GCC GAC ATG CTC ATG TA | |
| MK167 | Forward | 32 | TAA TGA GAG TGA GGG AAT ACC TTT G | 87 |
| | Taqman | 33 | FAM-GGC GTG TGT CCT TTG | |
| | probe | | GTG GGC A-BHQ1 | |
| | Reverse | 34 | AGG CAA GTT TTC ATC AAA TAG TTC A | |
| NPTN | Forward | 35 | ACC AGT GAA GAG GTC ATT ATT CGA GAC A | 88 |
| | Taqman | 36 | FAM-CCT GTT CTC CCT GTC | |
| | probe | | ACC CTG CAG TGT AAC-BHQ1 | |
| | Reverse | 37 | TAT GTA AGG GTG TGA GAG CTG GAG GT | |
| sNA12 | Forward | 38 | TGT GAC AAG GAA TAT GTG AGC CTG G | 81 |
| | Taqman | 39 | FAM-CCT GAA GAT GCA TAT TCG GAC | |
| | probe | | CCA CAC ATT-BHQ1 | |
| | Reverse | 40 | CGC AGA TCT TGC AAA CAC AAG G | |
| TERT | Forward | 41 | TGA CGT CCA GAC TCC GCT TCAT | 83 |
| | Taqman | 42 | FAM-GCT GCG GCC GAT | |
| | probe | | TGT GAA CAT GGA-BHQ1 | |
| | Reverse | 43 | ACG TTC TGG CTC CCA CGA CGT A | |
| VIM | Forward | 44 | ATG TTG ACA ATG CGT CTC TGG CA | 99 |
| | Taqman | 45 | FAM-TGA CCT TGA ACG | |
| | probe | | CAA AGT GGA ATC TTT GC-BHQ1 | |
| | Reverse | 46 | ATT TCC TCT TCG TGG AGT TTC TTC AAA | |
| GAPDH | Forward | 47 | CCA TCT TCC AGG AGC GAG ATC C | 90 |
| | Taqman | 48 | FAM-TCC ACG ACG TAC | |
| | probe | | TCA GCG CCA GCA-BHQ1 | |
| | Reverse | 49 | ATG GTG GTG AAG ACG CCA GTG | |

Table 5 is a list of primer and probe sequences for all markers used in the present invention.

### Comparative Example

Circulating tumor cells may exist in the blood in colorectal cancer or advanced adenoma, a precursor of colorectal cancer, and accordingly, an artificial intelligence algorithm-based model was constructed to determine the relative expression level of each group by targeting 10 genes (EpCAM, ERBB2, FOXA2, KRT19, MCAM, MKi67, NPTN, SNAI2, TERT, VIM)) known to have changes in relative expression level in circulating cancer cells, and to distinguish colorectal cancer or advanced adenoma from the normal group.

### Collection of Clinical Specimens

From 2017 to 2022, Blood samples from subjects scheduled for colonoscopy were collected at the Shinchon Severance Hospital (Approval No. 4-2017-0148), the Gangnam Severance Hospital (Approval No. 3-2017-0024), the Kangbuk Samsung Hospital (Approval No. 2017-02-022-009) in the Department of Gastroenterology, and the Health Examination Center of Wonju Severance Christian Hospital (approval number CR319115) with the approval of the Bioethics Review Board (RB) of each institution. A total of 3 ml of blood was collected using a Tempus blood tube (Applied Biosystems®). Subjects were classified as follows through the results of colonoscopy (Table 6)

TABLE 6

| | | No. of |
|---|---|---|
| | | samples |
| Classification | Classification criteria | (persons) |
| Colorectal | As a result of colonoscopy, | 148 |
| cancer | subjects with cancer in the colon | |
| group | | |
| Advanced | As a result of colonoscopy, | 289 |
| adenoma | subjects with advanced adenoma | |
| group | in the colon | |
| Normal | Subjects with no lesions in the | 142 |
| group | large intestine as a result of | |
| | colonoscopy | |

| | |
|---|---|
| Total | 579 |

Table 6 shows the classification of subjects and the number of samples according to colonoscopy results.
Isolation of Total RNA from Blood Specimens

Total RNA is isolated from a blood sample collected with a Tempus tube using the Tempus blood RNA isolation kit (Applied Biosystems®).

cDNA Construction from Isolated Total RNA and gPCR
i. Complementary DNA (cDNA) Synthesis
Isolated total RNA 1.5~4.5 ug, Random primer (3 ug/uL) (Invitrogen) 2.5 uL, dNTP mixture (2.5 mM each) (Intron) 2.5 uL, M-MLV reverse transcription polymerase (200 U/uL) (Invitrogen) 2.5 uL, 10 μL of 5× First-strand buffer (250 mM Tris-HCl) (Invitrogen), and 5 μL of Dithiothreitol (0.1 M) (Invitrogen) were added, and ultrapure water was added to a final volume of 50 μL, and mixed well. The synthetic reaction solution was reacted in a thermocycler (Applied Biosystems) at 25° C., 30 minutes—37° C., 50 minutes—70° C., 15 minutes to synthesize cDNA.
ii. Perform Quantitative Polymerase Chain Reaction (qPCR)
For the composition of the qPCR reaction, added 10 μL of THUNDERBIRD®Probe qPCR Mix (TOYOBO), Forward/Reverse Primer, Probe (10 pmole/uL) 1 μL, and added 2 μL of synthesized cDNA, and add ultrapure water to make the final volume 20 μL, and mixed. The qPCR reaction was performed using CFX96 (Biorad), and the reaction temperature conditions were as follows. After 95° C., 3 minutes, 95° C., 3 seconds—60° C., 30 seconds were repeated 40 times. Each time the annealing process (60° C., 30 seconds) was performed, a process of measuring fluorescence was added to measure the fluorescence value that increased by number of times. A constant fluorescence value was set as the threshold, and the Cq value, which is the number of cycles at the time of reaching the threshold, was derived.

### Confirmation of Results and Analysis of Relative Expression of Target Genes

Using the Cq value of the GAPDH gene used as an endogenous control, the relative expression level (2-ΔCq) of the target gene is calculated using the Cq value of the target gene. A list of targeted genes follows (Table 7).

$${2}^{- Δ Cq} = {2}^{- ( target gene Cq - GAPDH gene Cq )} \qquad [ Calculation formula ]$$

TABLE 7

| No. | Blood genetic markers | |
|---|---|---|
| 1 | EpCAM | Epithelial Cell Adhesion Molecule |
| 2 | ERBB2 | Erb-B2 Receptor Tyrosine Kinase 2 |
| 3 | FOXA2 | Forkhead Box A2 |
| 4 | KRT19 | Keratin 19 |
| 5 | MCAM | Melanoma Cell Adhesion Molecule |
| 6 | MKi67 | Marker Of Proliferation Ki-67 |
| 7 | NPTN | Neuroplastin |
| 8 | SNAI2 | Snail Family Transcriptional Repressor 2 |
| 9 | TERT | Telomerase Reverse Transcriptase |
| 10 | VIM | Vimentin |

Table 7 is a list of target blood genetic markers of comparative example.

### Establishment of a Classification Model for the Purpose of Screening for Colorectal Cancer and Advanced Adenoma by Substituting the Relative Expression Level of Target Genes

An artificial intelligence algorithm-based classification model was constructed using the H2O package (version 3.32.1.3) of Statistical R software (version 3.6.3). The production of colorectal cancer and advanced adenoma diagnosis prediction models was based on Deep neural network (DNN), Generalized linear model (GLM), Random Forest (RF), and Gradient boosting machine (GBM) algorithms, and several types of models (GLM, RF, DNN, GBM, stacked ensemble (SE)) was performed by grafting Automated machine learning (AutoML) method to build a model suitable for data, but is not limited thereto.
By dividing the entire sample into a training set and a test set, and by substituting the results of the training set, an artificial intelligence algorithm-based classification model that can distinguish between the colorectal cancer group and the advanced glandular group compared to the normal group is constructed, and the performance of the built model is evaluated using the test set (FIG. 4).
When building a model using a training set, a 5-fold cross-validation technique is applied so that the training set is divided into 5 areas and so at the same time as learning the model, the performance of the model was verified using each area to build a high-performance model.
The performance of the artificial intelligence classification model was judged through the AUROC and AUPRC values of the training set and test set based on the AUROC and AUPRC values, which are representative performance indicators of the classification model. Among them, the model with the best performance was selected based on the performance of the new test set that was not used for model learning. The AUROC and AUPRC values of the GLM, DNN, GBM, and RF models built based on each algorithm and the SE model built through AutoML are as follows (Table 8). As a result, the AUROC and AUPRC indicators were the highest in the RF and GBM model based on the test set.

TABLE 8

| | Training set | | Test set | |
|---|---|---|---|---|
| Model | AUROC | AUPRC | AUROC | AUPRC |
| GLM | 0.91 | 0.96 | 0.86 | 0.96 |
| RF | 0.90 | 0.96 | 0.94 | 0.98 |
| DNN | 0.99 | 1.00 | 0.92 | 0.97 |
| GBM | 1.00 | 1.00 | 0.94 | 0.98 |
| AutoML | 0.98 | 0.99 | 0.91 | 0.97 |
| (SE) | | | | |

Table 8 shows AUROC and AUPRC performance indicators in the training set and test set.

As a result of confirming the sensitivity and specificity of each group in the RF model and the GBM model, the sensitivity for distinguishing the colorectal cancer group in the RF model was 81.8% and the sensitivity for distinguishing the advanced adenoma group was 86.4% (Table 9). The specificity for classifying the normal group was 83.3%, the sensitivity for classifying the colorectal cancer group in the GBM model was 78.4%, the sensitivity for classifying the advanced adenoma group was 88.9%, and the specificity for classifying the normal group was 80.6% (Table 10). Therefore, an RF model with higher sensitivity for distinguishing colorectal cancer and higher specificity for distinguishing normal group was selected.

TABLE 9

| | Result of Test set | | | |
|---|---|---|---|---|
| | (Total 154 persons) | | | |
| | Positive | Negative | Sensitivity | Specificity |
| Classification | (persons) | (persons) | (%) | (%) |
| Colorectal | 100 | 18 | 84.7 | |
| cancer | | | | |
| group + | | | | |
| Advanced | | | | |
| adenoma | | | | |
| group(n = 118) | | | | |
| Colorectal | 30 | 7 | 81.1 | |
| cancer | | | | |
| group (n = 37) | | | | |
| Advanced | 70 | 11 | 86.4 | |
| adenoma | | | | |
| group(n = 81) | | | | |
| Normal | 6 | 30 | | 83.3 |
| group | | | | |
| (n = | | | | |
| 36) | | | | |

Table 9 shows the sensitivity and specificity results for each group of the RF model.

TABLE 10

| | Result of Test set | | | |
|---|---|---|---|---|
| | (Total 154 persons) | | | |
| | Positive | Negative | Sensitivity | Specificity |
| Classification | (persons) | (persons) | (%) | (%) |
| Color | 101 | 17 | 85.6 | |
| ectal cancer | | | | |
| group + | | | | |
| Advanced | | | | |
| adenoma | | | | |
| group(n = 118) | | | | |
| Colorectal | 29 | 8 | 78.4 | |
| cancer group | | | | |
| (n = 37) | | | | |
| Advanced | 72 | 9 | 88.9 | |
| adenoma | | | | |
| group(n =81) | | | | |
| Normal | 7 | 29 | | 80.6 |
| group(n = | | | | |
| 36) | | | | |

Table 10 shows the sensitivity and specificity results for each group of the GBM model.

1. A method for increasing the likelihood of making a positive colorectal cancer diagnosis, comprising:
a) separating a total RNA from cells obtained from a specimen;
b) synthesizing cDNA from a separated total RNA;
c) performing a quantitative polymerase chain reaction (qPCR) for the synthesized cDNA by using a primer set and a fluorescence-labeled probe capable of amplifying and detecting MKi67, KRT19, and EpCAM genes; a primer set and a fluorescence-labeled probe capable of amplifying and detecting TYMS, PPARG, MCAM and ANKHD1-EIF4EBP3 genes; and a primer set and a fluorescence-labeled probe capable of amplifying and detecting glyceraldehyde 3-phosphate dehydrogenase (GAPDH) as a control gene;
d) measuring fluorescence during the qPCR process; and
e) obtaining a relative expression level by measuring the fluorescence,
wherein the relative expression level is expressed as 2^{-ΔCq}, and 2^{-ΔCq} can be calculated using the following equation:
$${2}^{- Δ Cq} = {2}^{- ( target gene Cq - GAPDH gene Cq )} \qquad [ Calculation formula ]$$
wherein the target genes are MKi67, KRT19, EpCAM, TYMS, PPARG, MCAM, ANKHD1-EIF4EBP3 and the Cq value represents the cycle number at which a certain fluorescence value is set as a threshold and this threshold is reached,
wherein the expression level of TYMS, PPARG, MCAM, and ANKHD1-EIF4EBP3 genes in the specimen that is higher than the expression level of the MKi67, KRT19 and EpCAM genes in the specimen increases the likelihood that a specimen also having a detected expression level of TYMS, PPARG, MCAM, and ANKHD1-EIF4EBP3 genes is indicative of a positive colorectal cancer diagnosis.
